(19) United States
(12) Patent Application Publication
(10) Pub. No.: US 2024/0093278 A1
(43) Pub. Date: Mar. 21, 2024

(54) RAPID MOLECULAR DIAGNOSTICS WITH UNIFIED-ONE-POT SAMPLE PROCESSING, NUCLEIC ACID AMPLIFICATION, AND RESULT READOUT

(71) Applicant: DOMUS DIAGNOSTICS, INC., Park City, UT (US)

(72) Inventors: Xin Song, Raleigh, NC (US); Jacquelyn M. Walejko, Durham, NC (US); John H. Reif, Durham, NC (US)

(21) Appl. No.: 18/370,117

(22) Filed: Sep. 19, 2023

Related U.S. Application Data
(60) Provisional application No. 63/408,020, filed on Sep. 19, 2022.

(51) Int. Cl. C12Q 1/6844; C12Q 1/6806

(52) U.S. Cl. CPC C12Q 1/6844; C12Q 1/6806

(57) ABSTRACT

Disclosed herein are methods, compositions, and kits for rapid molecular diagnostics that integrate sample processing (including sample inactivation, sample lysis, nucleases inhibition, nucleic acid extraction, nucleic acid stabilization), nucleic acid amplification, and result readout into a single unified-one-pot reaction, enabling the molecular assay to be completed with minimal steps inside a single reaction vessel.

### CROSS-REFERENCE TO RELATED APPLICATIONS

This application claims priority to and the benefit of U.S. Provisional Patent Application No. 63/408,020, filed Sep. 19, 2022, and titled METHODS AND COMPOSITIONS FOR RAPID MOLECULAR DIAGNOSTICS WITH UNIFIED-ONE-POT SAMPLE PROCESSING, NUCLEIC ACID AMPLIFICATION, AND RESULT READOUT, which is incorporated herein in its entirety by reference.

### BACKGROUND

Establishing widespread access to rapid, accurate, affordable testing is a critical part of pandemic preparedness. Recent years have witnessed a dramatic increase in the research and development of alternative nucleic acid detection techniques that are simple, low-cost, and better suited for point-of-care (POC) and at-home diagnostic applications without reliance on specialized instrumentation and trained personnel.

One such exemplary protocol is loop-mediated isothermal amplification (LAMP, or RT-LAMP if the target is RNA), which uses a set of four to six primers and a strand-displacing DNA polymerase (or in addition, a reverse transcriptase if the target is RNA) to exponentially amplify trace amount of the target nucleic acid under isothermal conditions (i.e., reactions take place at a constant temperature without the need for thermocycling). Such molecular tests are generally more sensitive and accurate than rapid antigen tests for the diagnosis of infectious diseases. However, despite being more tolerant to inhibitors than the conventional polymerase chain reaction (PCR), the performance of isothermal amplification techniques such as LAMP and RT-LAMP still largely depends on the quality of samples, and conventional approaches typically require dedicated pre-processing via a combination of thermal, mechanical, and/or chemical treatments for sample inactivation, lysis, nucleases inhibition, nucleic acid extraction, and purification in separate steps before the sample is prepared for a downstream amplification reaction.

Accordingly, there remains an ongoing need for improved techniques directed to isothermal nucleic acid amplification assays.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification.

FIG. 1 compares different molecular testing workflows. (A) Conventional nucleic acid extraction and amplification workflow. (B) Simplified protocol (2-step, 2-temperature) from sample to result. (C) Unified-one-pot protocol (1-step, 1-temperature) from sample to result according to the present disclosure.

FIG. 2 illustrates results of a unified-one-pot assay based on exemplary lysis buffers formulated with TBE or EDTA.

FIG. 3 illustrates results of a unified-one-pot assay based on exemplary lysis buffers formulated with VSA.

FIGS. 4-6 illustrates results of a unified-one-pot assay performance based on exemplary lysis buffers formulated with formamide.

FIG. 7 illustrates results of a unified-one-pot assay performance based on exemplary formamide and urea lysis buffer formulations.

FIG. 8 illustrates results of a unified-one-pot assay performance based on exemplary urea lysis buffer formulations.

FIG. 9 illustrates results using urea lysis buffer for unified-one-pot assay with self-collected nasal swabs.

FIG. 10 illustrates results of a unified-one-pot assay for different targets, with Flu A, Flu B, and RSV virus spiked in optimized urea lysis buffer with RNase A added.

FIG. 11 illustrates results of a unified-one-pot assay for different targets using one-pot lyophilized beads and optimized urea lysis buffer.

FIG. 12 illustrates results of unified-one-pot assays based on different RT-LAMP master mixes and colorimetric readout indicators.

FIG. 13 illustrates results of a unified-one-pot assay based on different RT-LAMP reaction mechanisms, including pH-dependent and pH-independent mechanisms.

FIG. 14 illustrates results of unified-one-pot assays carried out using an example assay device for simultaneous assaying of different targets with one-pot lyophilized beads and urea lysis buffer.

FIG. 15 illustrates stability performance of the unified-one-pot assay using the example assay devices comprising one-pot lyophilized beads and sample collection tubes prefilled with lysis buffer.

### DETAILED DESCRIPTION

### Overview

Certain embodiments of this application include or incorporate features described in: International Application No. PCT/US2022/021138, published as WO 2022/204023; U.S. application Ser. No. 17/749,858, published as US 2022/0372569; and/or U.S. application Ser. No. 18/116,138, published as US 2023/0279478, each of which is hereby incorporated by reference in its entirety.

To reduce the cost and complexity of molecular testing workflows for POC and at-home usage, provided herein are methods, compositions, and kits for rapid molecular diagnostics that integrate the sample processing (including but not limited to sample inactivation, sample lysis, nucleases inhibition, nucleic acid extraction, nucleic acid stabilization), nucleic acid amplification, and result readout into a single unified-one-pot reaction, thus enabling the molecular assay to be easily completed with minimal steps inside a single reaction vessel. Applications of the methods and compositions described herein include but are not limited to the detection of pathogens, diagnostics of cancers and infectious diseases, SNP genotyping, quality control of food and dietary products, and environmental surveillance.

Throughout this disclosure, certain examples are described in the context of RNA as the target nucleic acid. It will be understood that similar components and method steps may be readily utilized for assays targeting DNA with minimal modification (e.g., removal of reverse transcriptase). The converse is also true. That is, example assays describing DNA as the target nucleic acid can be readily adapted to target RNA with minimal modification (e.g., the addition of reverse transcriptase). Similarly, examples describing RT-LAMP can also be adapted to LAMP, and vice versa, as appropriate for the type of nucleic acid targeted by the assay.

### Example Unified-One-Pot Methods

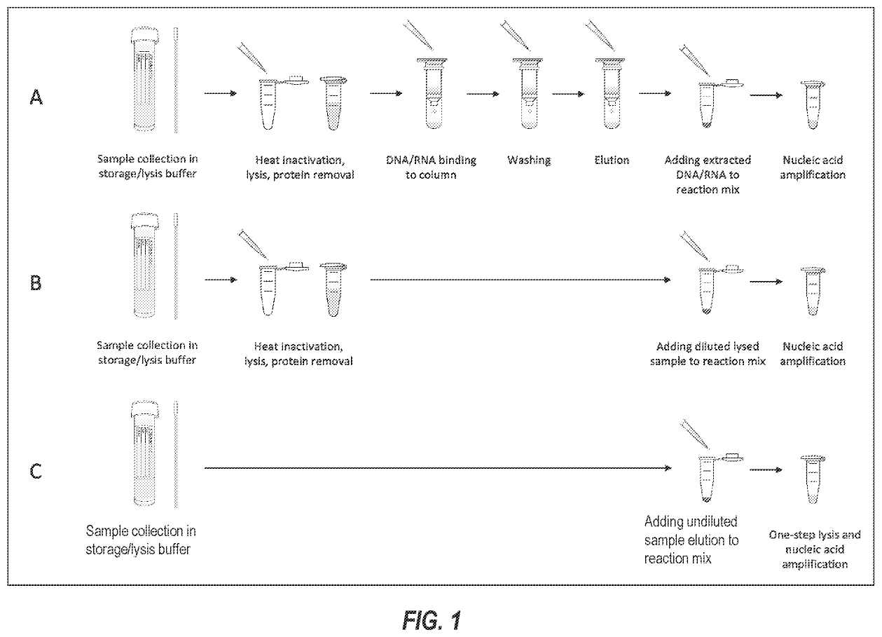

FIG. 1 presents an illustrative schematic comparing different molecular testing workflows. (A) shows an example of a conventional nucleic acid extraction and amplification workflow, which involves sample processing through a series of steps requiring multiple different reagents, liquid transfers, and lab equipment such as centrifuge and pipettes. (B) is an illustrative example of a simplified protocol (2-step, 2-temperature) from sample to result. Such a protocol eliminates the time-consuming RNA extraction steps, but still requires a separate lysis step typically based on high temperature (e.g., 95° C.) requiring the use of freshly prepared lysing agents and can only allow a relatively small amount of the mixture comprising the lysed sample (e.g., 1-5 μL) to be added into the subsequent nucleic acid reaction (e.g., 20-25 μL, reaction volume) due to inhibitory effects of the lysing agents.

(C) is an exemplary illustration of the presently disclosed unified-one-pot protocol (1-step, 1-temperature) from sample to result. In this protocol, a portion of the crude sample elution (sample eluted in a sample processing buffer) can be directly added into a nucleic acid amplification reaction mix (e.g., prepared in wet, frozen, air dried, or lyophilized format) to enable a unified-one-pot reaction to take place, which accomplishes both the sample processing (including but not limited to sample inactivation, sample lysis, nucleases inhibition, nucleic acid extraction, nucleic acid stabilization) and nucleic acid amplification (e.g., LAMP, RT-LAMP) by simply incubating the closed reaction vessel at a single temperature (e.g., 60-68° C.) for a short period of time (e.g., 15-45 minutes). Compared to (B), protocol (C) can be simpler, faster, lower cost, and allows for a relatively larger sample collection volume to be directly added to the amplification reaction without further dilution owing to the non-inhibitory nature of the sample processing buffer. The ability to add larger sample volumes to the amplification reaction beneficially improves ease of use and sensitivity of the assay. For example, a larger volume of sample mixture (e.g., greater than 5 μL) can be transferred to the amplification reaction vessel to provide a greater total amount of target nucleic acid to the reaction, thereby increasing the sensitivity of the assay relative to the volume-limited approach of (B).

In some embodiments, the sample can be a lower nasal swab sample, nasopharyngeal swab sample, gingival swab sample, buccal swab sample, gargle sample, sputum sample, or saliva sample. In some embodiments, the sample may include other bodily fluids depending on the types of diseases, biomarkers, or pathogens specific to the assay. In some embodiments, the sample can be an environmental sample collected from soil or water, for example. In some embodiments, the sample can be agricultural or food products such as feedstocks, vegetables, fruits, meat, milk, honey, etc. In some embodiments, the target of the molecular diagnostic test may be viruses, bacteria, algae, or other types of microorganisms.

In some embodiments, the system described herein may be configured to involve first collecting a sample, then eluting the sample into a solution (e.g., sample processing buffer) that is formulated to effectively inactivate and lyse the sample (e.g., viral particles) and stabilize the released nucleic acid material under proper conditions (including but not limited to temperature, pH, buffer composition) that are also compatible for a simultaneous nucleic acid amplification reaction to take place in the same reaction mix without inhibition or cross-reactivity.

In some embodiments, the method comprises use of a sample processing buffer, an RT-LAMP master mix, and end-point/real-time readout chemistry, which are together compatible in a unified-one-pot format such that after collecting a sample into the sample processing buffer, a portion of the “sample mixture” (i.e., the mixture resulting from adding the sample to the sample processing buffer) can be directly added (manually or by any suitable sample transfer mechanisms such as microfluidics) into an RT-LAMP reaction mix (also referred to herein as a “master mix”). The master mix can be prepared in wet, frozen, air dried, or lyophilized form. Addition of the sample mixture to the master mix initiates a one-pot reaction that allows both the sample processing (including but not limited to sample inactivation, sample lysis, nucleases inhibition, nucleic acid extraction, nucleic acid stabilization) and the nucleic acid amplification (e.g., LAMP, RT-LAMP) to take place within the same reaction vessel by incubation at a single temperature (e.g., 60-68° C.) for a short period of time (e.g., 15-45 minutes). Subsequently, the result of the test can be read (without the need to open the reaction vessel) by direct naked-eye (colorimetric) or device-assisted (e.g., colorimetric, fluorescent, or electrochemical, using an analysis device, such as a handheld computer device, such as a smartphone) interpretation. In some embodiments, the result may be qualitative (yes-or-no) based on a binary readout, whereas in some other embodiments, the readout result may be semi-quantitative or quantitative.

The disclosed embodiments can therefore beneficially omit a separate heating step for sample inactivation and lysis. That is, the disclosed embodiments do not require a step of heating the sample collection media (usually carried out at approximately 95° C. in prior methods). Instead, the sample processing buffer functions as a sample collection medium that is capable of being mixed with the master mix and incubated at a single temperature (e.g., 60-68° C.). Unlike prior approaches, the disclosed embodiments beneficially avoid the need for a separate inactivation/lysing heating step and the immediately subsequent cooling step prior to transfer of the sample collection medium to the master mix components for amplification. This saves processing time, reduces the number of workflow steps, simplifies the process, and therefore reduces the risk of user error or other unintentional process irregularities.

In some embodiments, the disclosed methods and compositions for unified-one-pot chemistry may be integrated with any suitable systems or devices or combinations thereof designed for sample collection, reagent storage, fluid manipulation, thermal incubation, and result readout and analysis.

In some embodiments, a method for performing a molecular diagnostic test that combines sample processing, nucleic acid amplification, and result readout in a single unified-one-pot reaction comprises: mixing a sample with a sample processing buffer to form a sample mixture; adding the sample mixture or portion thereof directly to a nucleic acid amplification master mix within a reaction volume to form a reaction mixture; and subjecting the reaction mixture within the reaction volume to thermal incubation for a period of time, during which (i) the sample is processed, (ii) target nucleic acids are released and amplified, and (iii) a readout indicator is activated.

The method can omit a step of heating the sample mixture prior to adding the sample mixture to the nucleic acid amplification master mix. The thermal incubation following addition of the sample mixture or portion thereof to the master mix can include raising the reaction volume to a temperature no greater than about 85° C., such as no greater than about 80° C., such as no greater than about 75° C., such as no greater than about 70° C., such as to a temperature of about 60-68° C. The reaction volume can be contained within any suitable container. In some embodiments, the reaction volume is disposed within a reaction chamber as part of a microfluidic assay device.

The sample mixture or portion thereof can be added to the nucleic acid amplification master mix by any suitable fluidic transfer mechanism, including but not limited to pipetting, microfluidics, microcapillaries, wicking by a porous media or material, or combinations thereof, in either manual, semi-automatic, or automatic fashion.

In some embodiments the nucleic acid amplification master mix is in an air-dried or lyophilized form. In such embodiments, the sample mixture or portion thereof, when added to the master mix, can directly reconstitute the reaction mixture to a desired total reaction volume.

In some embodiments, the nucleic acid amplification is isothermal, and the unified-one-pot reaction takes place inside a single reaction vessel at a single target (i.e., set) incubation temperature. In alternative embodiments, the nucleic acid amplification is not isothermal, and the unified-one-pot reaction takes place inside a single reaction vessel subjected to suitable thermocycling conditions. A “single target incubation temperature” refers to a single intended temperature during the relevant portion of the assay workflow (e.g., during the amplification reaction). The “single target incubation temperature” can still include changes during warm up and cool down, and can still include relatively small temperature variations due to ambient fluctuations, heating instrument tolerances, and/or insulative properties of the reaction vessel, for example, so long as the intent is to substantially maintain a single desired temperature.

### Example Kits

The present disclosure also relates to a kit for performing a molecular diagnostic test in a unified-one-pot format. The kit can comprise: a sample collection device; a first vessel that contains a sample processing buffer; a second vessel that contains a frozen, air-dried, or lyophilized nucleic acid amplification master mix formulated for nucleic acid amplification and result readout; and a device for transferring a portion of a sample mixture from the first vessel into the second vessel. The kit can optionally further include a heat source configured to generate heat at a temperature profile suitable for performing a unified-one-pot sample processing and nucleic acid amplification reaction. The kit can optionally further include a reader device to aid in interpretation and report of the test result (e.g., qualitatively, semi-quantitatively, or quantitatively) in a semi- or fully automatic fashion.

The heat source can be internally powered or externally powered, and may be battery-powered, USB-powered, solar-powered, chemically powered by an exothermic reaction, and variations and combinations thereof. The kit can further comprise a mechanism for regulating the output temperature profile. For example, the kit can include an insulative container configured with sufficient insulation to maintain a desired output temperature profile for effectively carrying out the unified-one-pot reaction.

### Example Amplification Methods

The target nucleic acid may be amplified by any suitable nucleic acid amplification methods including but not limited to isothermal amplification methods' such as LAMP, RT-LAMP, dual-priming isothermal amplification (DAMP), cross-priming amplification (CPA), strand displacement amplification (SDA), rolling circle amplification (RCA), recombinase polymerase amplification (RPA), helicase-dependent amplification (HDA), nucleic acid sequence-based amplification (NASBA), multiple displacement amplification (MDA), whole genome amplification (WGA), genome exponential amplification reaction (GEAR), exponential amplification reaction (EXPAR), nicking and extension amplification reaction (NEAR), single chimeric primer isothermal amplification (SPIA), isothermal and chimeric primer-initiated amplification of nucleic acid (ICAN), hairpin fluorescence probe-assisted isothermal amplification (PHAMP), signal-mediated amplification of RNA technology (SMART), beacon-assisted molecular detection (BAD AMP), CRISPR-Cas9-triggered nicking endonuclease-mediated strand displacement amplification (CRISDA), as well as enzyme-free nucleic acid amplification methods' such as hybridization chain reaction (HCR), catalyzed hairpin assembly (CHA), exponential hairpin assembly (EHA), entropy-driven catalysis (EDC) such as toehold-mediated strand displacement (TMSD), and variations and combinations thereof.

The amplification process, either enzymatic or non-enzymatic, may leverage any suitable catalytic or autocatalytic 5 reaction mechanisms to achieve signal amplification, wherein catalytic reactions refer to reactions with the presence of at least one catalyst to increase reaction rates, and autocatalytic reactions refer to reactions in which at least one of the products is also a reactant or catalyst for the same reaction or a coupled reaction.

In some cases, the unified-one-pot chemistry may also be broadly adapted for use with conventional thermocycling-based methods such as PCR, RT-PCR, real-time qPCR/RT-qPCR, and variations thereof.

Sample Processing Buffer

The sample processing buffer may include one or more compounds and/or enzymatic components for sample elution, inactivation, lysis, nucleases inhibition, nucleic acid extraction, and/or stabilization. A sample processing buffer may include, for example: nuclease-free water; a surfactant such as polysorbate 20 (trade name: Tween 20), polysorbate 80 (trade name: Tween 80), polyethylene glycol p-(1,1,3,3-tetramethylbutyl)-phenyl ether (trade name: Triton X-100), 1,1,3,3-Tetramethylbutyl)phenyl-poly ethylene glycol, (trade name: Triton X-114), octylphenoxypolyethoxyethanol (trade name: Nonidet P-40 or NP-40), branched octylphenoxypolyethyleneoxyethanol (trade name: Igepal CA-630), 3-1(3-cholamidopropyl)dimethylammoniol-1-propanesulfonate (CHAPS), and/or sodium dodecyl sulfate (SDS); a reducing/denaturing agent such as dithiothreitol (DTT), tris(2-carboxyethyl)phosphine (TCEP), urea, guanidine hydrochloride (GuHCl), guanidinium thiocyanate (GITC), and/or formamide;⁶⁻⁸ a nuclease inhibitor such as proteinase K, murine RNase inhibitor, human placenta RNase inhibitor, vinylsulfonic acid (VSA), polyvinylsulfonic acid (PVSA),^{9,10} achromopeptidase (ACP), native or recombinant nuclease inhibitors such as those sold under the trade names RNasin Ribonuclease Inhibitor, RNasin Plus Ribonuclease Inhibitor, RiboLock RNase Inhibitor, SUPERaseIn, RNaseOUT, and/or RNAsecure. The sample processing buffer may further comprise one or more of: chelating agents such as ethylenediaminetetraacetic acid (EDTA); and/or buffering salts such as tris(hydroxymethyl)aminomethane (commonly referred to as Tris), Tris-HCl, Tris-EDTA (TE), Tris-acetate-EDTA (TAE), Tris-borate-EDTA (TBE)¹¹, HCl solution, NaOH or KOH solution.

In some embodiments, one or more components typically included in the master mix (e.g., MgSO₄, MgCl₂, colorimetric indicator, etc.) may be additionally or alternatively included in the sample processing buffer. In some embodiments, one of more components typically included in the sample processing buffer (e.g., nuclease inhibitor, etc.) may be additionally or alternatively included in the master mix (e.g., in one-pot air-dried or lyophilized form) according to cost and/or stability considerations. For example, including one or more nuclease inhibitors in the master mix allows for stabilization via air drying or lyophilization.

In a presently preferred embodiment, the sample processing buffer may include the following, optionally included at the recited concentrations: a surfactant such as Tween 20 and/or Triton X-100 at a concentration ranging from about 0.025% to about 10%, or about 0.05% to about 7.5%, or about 0.075% to about 5%, or about 0.1% to about 2.5%, or about 0.1% to about 1%, or a value within a range with endpoints defined by any two of the foregoing values; reducing/denaturing agents including (1) formamide at a concentrations ranging from about 0.1% to 10%, or about 0.5% to about 8.5%, or about 1% to about 7%, or about 2% to about 6%, or a value within a range with endpoints defined by any two of the foregoing values, and/or (2) urea at a concentration ranging from about 0.5 mM to 1.8 M, or about 5 mM to 1M, or about 15 mM to 500 mM, or about 25 mM to 250 mM, or about 50 mM to 150 mM, or a value within a range with endpoints defined by any two of the foregoing values; nuclease inhibitors including VSA or PVSA at a concentration from about 0.01 mg/mL to 10 mg/mL, or about 0.05 mg/mL to 5 mg/mL, or about 0.1 mg/mL to 1 mg/mL, or a value within a range with endpoints defined by any two of the foregoing values; and/or murine RNase inhibitor or human placenta RNase inhibitor or RNasin Ribonuclease Inhibitor (native or recombinant) or RNasin Plus Ribonuclease Inhibitor or RiboLock RNase Inhibitor or RNaseOUT at a concentration ranging from about 0.05 units/μL to 2 units/μL, or about 0.1 units/μL to 1 units/μL, or a value within a range with endpoints defined by any two of the foregoing values. As discussed above, in some embodiments, one or more nuclease inhibitors may be additionally or alternatively included in the master mix.

The sample processing buffer can optionally further comprise: a buffering salt such as TBE at a concentration ranging from about 0.001× to about 1×, or about 0.0015× to about 0.5×, or about 0.002× to about 0.25×, or about 0.0025× to about 0.1×, or a value within a range with endpoints defined by any two of the foregoing values; and/or a chelating agent such as EDTA at a concentration ranging from about 0.005 mM to 2.5 mM, or about 0.05 mM to about 1.5 mM, or about 0.5 mM to about 1 mM, or a value within a range with endpoints defined by any two of the foregoing values. In some cases, the sample processing buffer is adjusted to pH about 8.0-8.2 for optimal compatibility with the nucleic acid amplification master mix.

### Amplification Master Mix

In some embodiments, such as where RT-LAMP is used as the nucleic acid amplification method, the master mix may be formulated for either pH-dependent or pH-independent detection of the target nucleic acid. The master mix may comprise one or more of the following components (with recited concentrations representing examples only): 4 mM to 8 mM MgSO₄ or MgCl₂; 10 mM (NH₄)₂SO₄ or (NH₄)₂C12; 1 mM to 2 mM dNTP mix; DNA polymerase (e.g., 0.32 U/4 Bst 2.0 or Bst 2.0 WarmStart DNA Polymerase); 0.1% to 2% Tween 20 or Triton-100 (e.g., pH 8.8); 2 mM to 20 mM Tris-HCl (pH 8.8) or Tris or TE at suitable concentration; and 10 mM to 50 mM KCl.

According to application needs, the master mix may further comprise one or more of primers (e.g., 0.8 μM F3 primer, 0.8 μM B3 primer, 1.6 μM FIP primer, 1.6 μM BIP primer, and in some cases further comprising 0.4 μM LF primer and/or 0.4 μM LB primer); reverse transcriptase (e.g., 0.2 U/4 WarmStart RTx Reverse Transcriptase); 0.8 mM betaine; 10 mM to 60 mM GuHCl; and/or Antarctic Thermolabile Uracil-DNA-glycosylase (UDG) and dUTP to prevent carryover contamination.

According to application needs, the master mix may further comprise one or more additives known in the art to enhance the performance (e.g., sensitivity, specificity, speed, robustness) of nucleic acid amplification reactions, including but not limited to: crowding agents (e.g., polyethylene glycol (PEG), Ficoll, dextran), dsDNA destabilizers (e.g., helicase, recombinases, endonucleases, ionic liquids, proline), dsDNA stabilizers (e.g., Tetramethylammonium chloride (TMAC)), enzyme stabilizers (e.g., bovine serum albumin (BSA), trehalose, pullulan), template blockers (e.g., single-stranded DNA-binding proteins (SSBs), graphene oxide (GO), cobalt oxyhydroxide (CoOOH) nanoflakes), and/or oligonucleotide modifications or analogs (e.g., locked nucleic acids (LNA), and/or phosphorothioate nucleotide analogues (PTO), peptide nucleic acids (PNA)).

The reaction mix may include one or more excipients (including but not limited to sucrose, trehalose, dextran, pullulan, lactose, glucose, raffinose, mannitol, sorbitol, glycine, histidine, arginine, gelatin, dextrose, hydroxyethyl starch, poly(ethylene glycol), poly(propylene glycol), poly(vinyl alcohol), or combinations thereof) to form stabilized reaction mixes (by techniques such as air drying or lyophilization) to enable extended storage at ambient conditions.

### Readout Indicator

The readout indicator may be included in the sample processing buffer and/or the master mix according to, for example, stability requirements and/or other application parameters.

The readout indicator can include: a pH indicator such as phenol red, neutral red, cresol red, cresol purple, thymol blue, bromothymol blue, bromophenol blue, litmus, chlorophenol red, dichlorofluorescein, methyl red, bromocresol purple, naphtholphthalein, and/or cresolphthalein; a metal indicator that senses metal ions such as M g²⁺, Mn²⁺, Zn²⁺, Cu²⁺, Co²⁺, Fe²⁺, Ni²⁺, Hg²⁺, Pb²⁺, such as a composition comprising one or more of hydroxynaphthol blue, eriochrome black T, calcein, or pyridylazophenol dye such as 2-(5-Bromo-2-pyridylazo)-54N-propyl-N-(3-sulfopropyl) aminolphenol (5-Bromo-PAPS) or 2-(5-Nitro-2-pyridylazo)-5-1N-n-propyl-N-(3-sulfopropyl)amino] phenol (5-Nitro-PAPS); a fluorescent/colorimetric DNA binding dye such as SYBR Gold, SYBR Safe, leuco crystal violet, malachite green, methyl green, EvaGreen, SYTO 9; and/or a nanoparticle-based indicator such as gold nanoparticles.

For example, in some embodiments, the reaction mix may include a pH indicator (e.g., 50 μM to 100 μM phenol red or alternative pH indicator 12 at suitable concentration), a metal indicator system (e.g., 0.12 mM hydroxynaphthol blue, 25 μM Calcein with 0.5 mM MnCl₂, 50 μM to 200 μM 5-Bromo-PAPS with 50 μM to 300 μM Mn²⁺, or alternative pyridylazophenol metal sensing dye 13-17 combined with suitable metal ion at suitable concentrations), and/or dsDNA intercalator (e.g., 0.004% malachite green^{18,19} or methyl green²⁰, or alternative dsDNA intercalator at suitable concentration). The foregoing concentration amounts are optional.

The reaction readout may involve mechanisms based on turbidity, 21 fluorescence (e.g., calcein, EvaGreen, GelGreen, GelRed, SYTO 9), nanomaterials (e.g., gold nanorods/nanoparticles), detection of pyrophosphate (e.g., utilizing metal ions with pyridylazophenol dyes), lateral flow strip/dipstick, gel/capillary electrophoresis, microfluidics, microarrays, electrochemical sensors, molecular transducers, or variations and combinations thereof.^{22,23}

### Examples

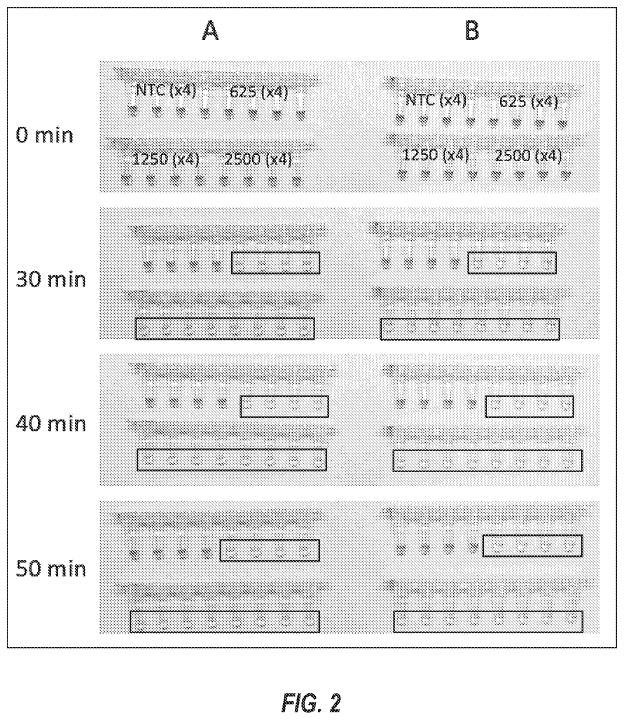

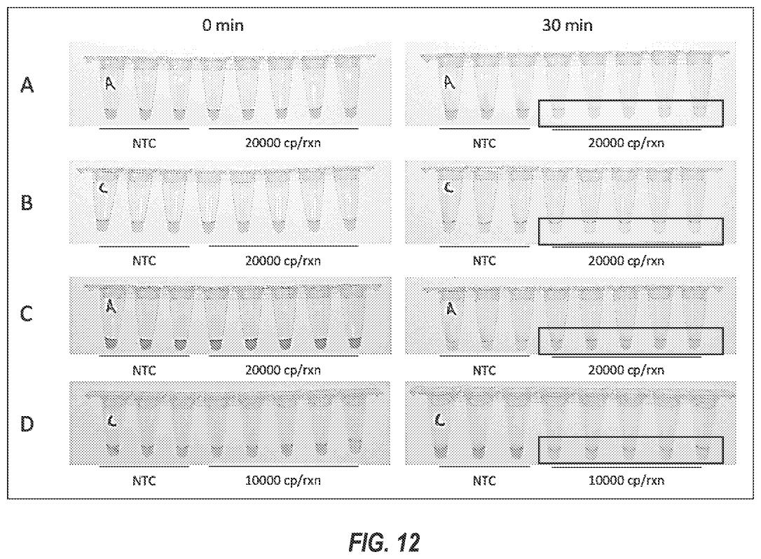

FIGS. 2-15 illustrate results of unified-one-pot assays using various sample processing buffers and that were carried out according to at least some of the concepts disclosed herein. The assays of FIGS. 2-11 utilized a phenol red indicator, which is a pH-dependent indicator that changes to yellow to indicate positive detection of the target nucleic acid. Assay (C) of FIG. 12 utilized a malachite green indicator, in which positive samples maintain color while negative samples become colorless. As disclosed above, other indicators may additionally or alternatively be utilized.

The grayscale photographs of the Figures do not fully illustrate the specific colors and color changes of the indicators. Accordingly, throughout the Figures, to better illustrate the resulting colorimetric readout on the grayscale photographs, the test tubes that clearly exhibited a positive result (indicating presence of the target nucleic acid) are enclosed by an indicator box.

FIG. 2 illustrates results of a unified-one-pot assay based on lysis buffers formulated with TBE or EDTA. Assay (A) included 0.0025× TBE+0.1% Tween 20. Assay (B) included 0.05 mM EDTA+0.1% Tween 20. Assays were carried out with simulated clinical samples (e.g., SARS-CoV-2 virus spiked in swab elution in respective lysis buffer). The non-template control (NTC) and virus copy number per reaction were as indicated. The master mix formulas of the RT-LAMP reactions were lyophilized in one-pot format prior to addition of simulated samples and testing.

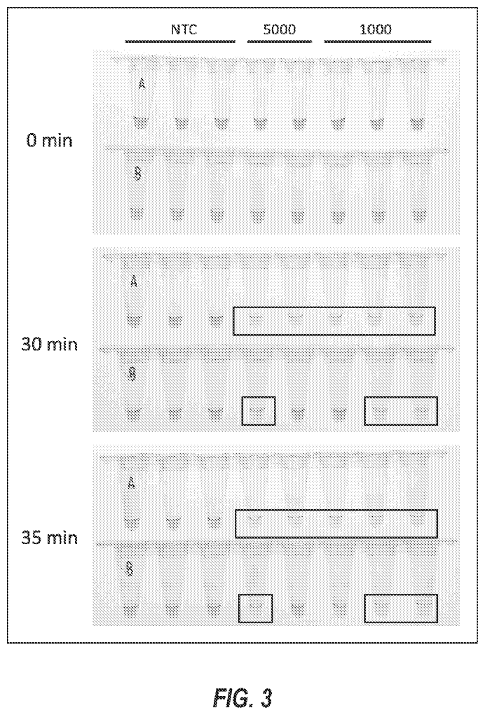

FIG. 3 illustrates results of a unified-one-pot assay based on lysis buffers formulated with VSA. Assay (A) included 60 μg/mL VSA+0.1% Tween 20. Assay (B) included 100 μg/mL VSA+0.1% Tween 20. Assays were performed with simulated clinical samples (e.g., SARS-CoV-2 virus spiked in swab elution in respective lysis buffer). The NTC and virus copy number per reaction were as indicated. The amplification reactions were based on wet RT-LAMP.

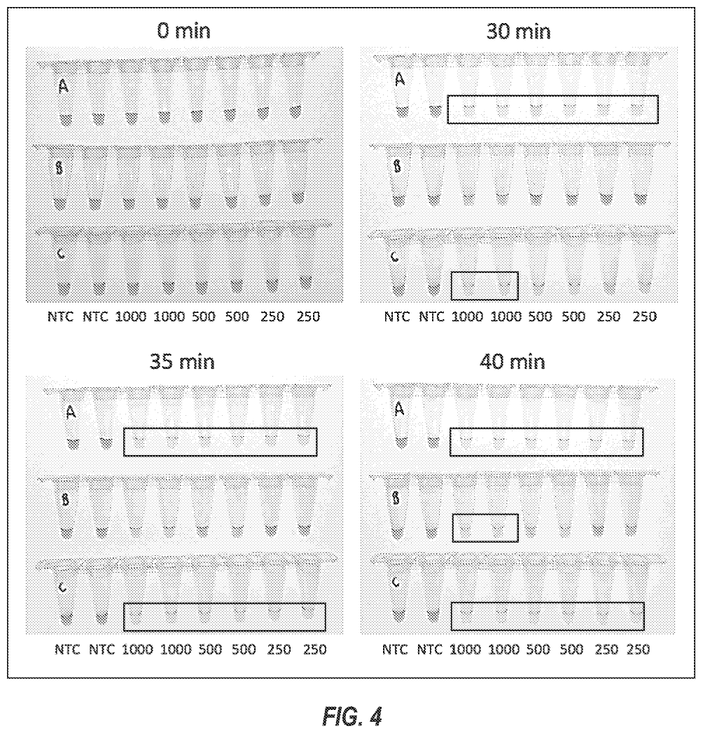

FIG. 4 illustrates results of a unified-one-pot assay based on lysis buffers formulated with formamide. Assay (A) included 6% formamide+0.1% Tween 20. Assay (B) included 6% formamide+0.1% Tween 20+50 μg/mL proteinase K. Assay (C) included 6% formamide+0.1% Tween 20+50 μg/mL proteinase K (followed by inactivation at 95° C. for 5 min). Assay (C) does not meet the requirement of unified-one-pot format but was included for comparison. Assays were performed with simulated clinical samples (e.g., SARS-CoV-2 virus spiked in swab elution in respective lysis buffer). The NTC and virus copy number per reaction were as indicated. The amplification reactions were based on wet RT-LAMP.

FIG. 5 illustrates results of a further unified-one-pot assay based on formamide lysis buffer formulations. Assay (A) included 6% formamide+0.1% Tween 20+2 U RNasin Plus. Assay (B) included 2% formamide+0.1% Tween 20 +2 U RNasin Plus. Assay (C) included 2% formamide+0.1% Tween 20+1 U RNasin Plus. Assay (D) included 2% formamide+0.1% Tween 20. Assays were performed with simulated swab samples (e.g., SARS-CoV-2 virus spiked in respective lysis buffer with RNase A added). The NTC and virus copy number per reaction were as indicated. The amplification reactions were based on wet RT-LAMP.

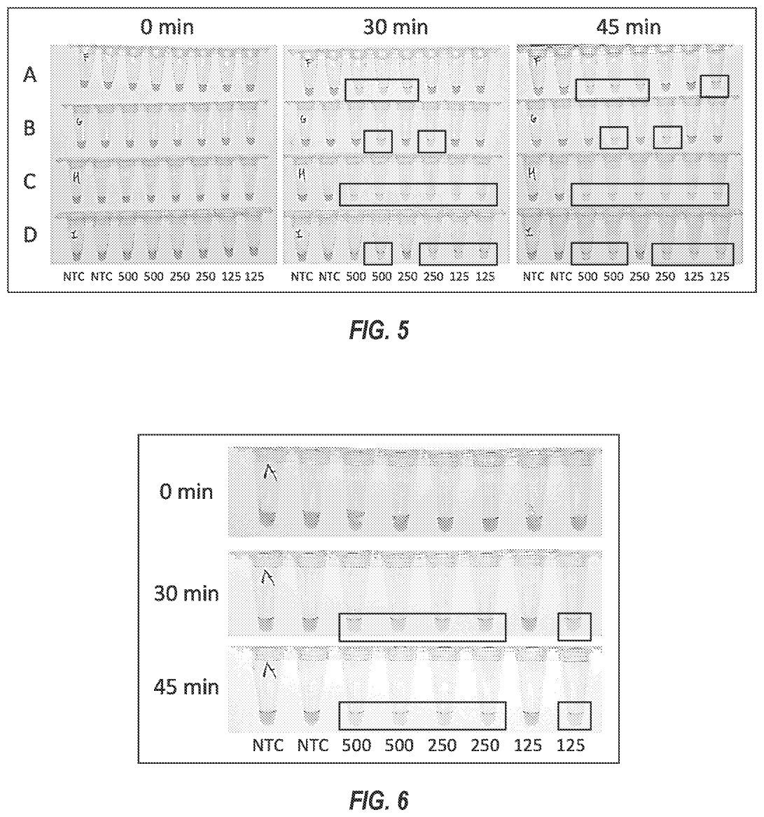

FIG. 6 illustrates results of a further unified-one-pot assay based on formamide lysis buffer formulation including 2% formamide+0.1% Tween 20+1 U RNasin Plus. Assays were performed with simulated swab samples (e.g., SARS-CoV-2 virus spiked in lysis buffer with RNase A added). The NTC and virus copy number per reaction were as indicated. The amplification reactions were based on one-pot RT-LAMP using a lyophilized master mix.

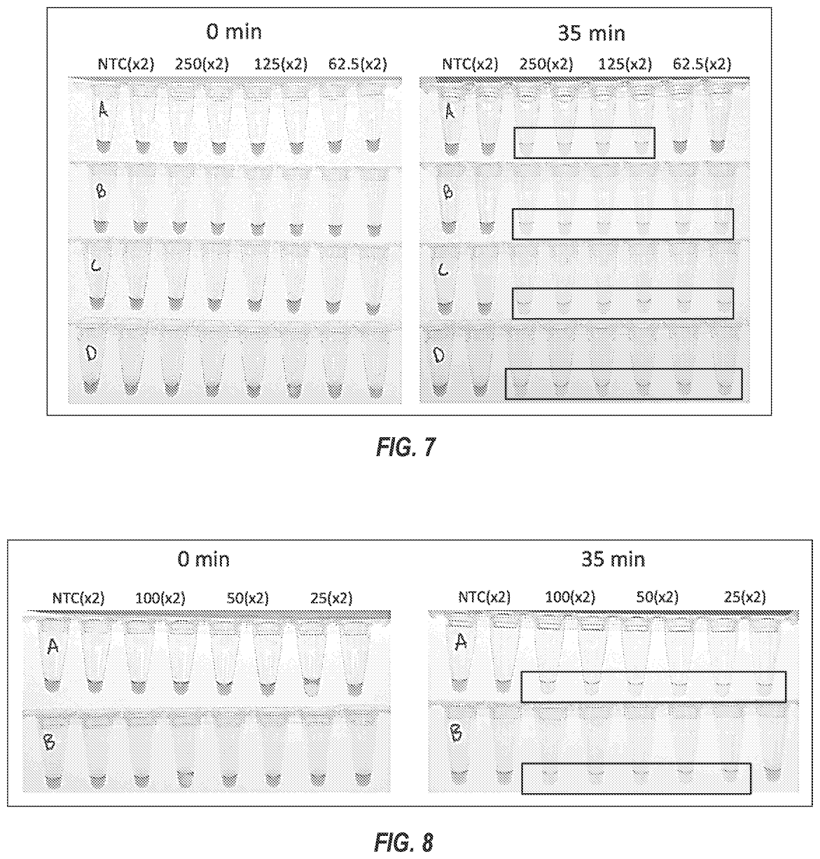

FIG. 7 illustrates results of a unified-one-pot assay based on formamide and urea lysis buffer formulations. Assay (A) included 2% formamide+0.1% Tween 20. Assay (B) included 25 mM urea+0.1% Tween 20. Assay (C) included 100 mM urea+0.1% Tween 20. Assay (D) included 400 mM urea+0.1% Tween 20. All assays included 0.1 U/μL RNasin Plus. Assays were performed at 10 uL final volume with simulated swab samples (e.g., SARS-CoV-2 virus spiked in lysis buffer with RNase A added). The NTC and virus copy number per reaction were as indicated. The amplification reactions were based on wet RT-LAMP.

FIG. 8 illustrates results of a further unified-one-pot assay based on urea lysis buffer formulations. Assay (A) included 10 mM urea+0.1% Tween 20. Assay (B) included 25 mM urea+0.1% Tween 20. All assays contained 0.1 U/μL RNasin Plus. Assays were performed at 10 uL final volume with simulated swab samples (e.g., SARS-CoV-2 virus spiked in lysis buffer with RNase A added). The NTC and virus copy number per reaction were as indicated. The amplification reactions were based on wet RT-LAMP.

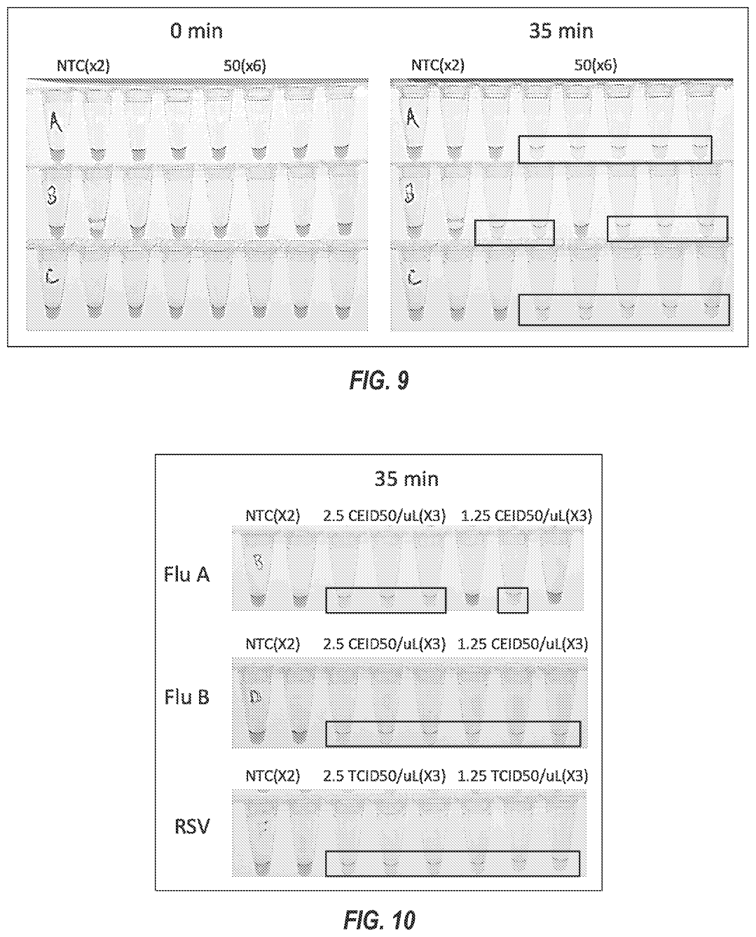

FIG. 9 illustrates results following further optimization and evaluation of urea lysis buffer for unified-one-pot assay with self-collected nasal swabs. Assay (A) included 5 mM urea+0.1% Tween 20. Assay (B) included 10 mM urea+0.1% Tween 20. Assay (C) included 25 mM urea+0.1% Tween 20. All assays contained 0.1 U/μL RNasin Plus. Assays were performed at 10 μL final volume with real nasal swabs. The NTC and virus copy number per reaction were as indicated. The amplification reactions were based on wet RT-LAMP. The sample processing buffers used in these assays beneficially exhibited improved sensitivity, a lower limit of detection, enhanced reproducibility, and effective buffer stability, particularly the sample processing buffer used in assay (B).

FIG. 10 illustrates results of a unified-one-pot assay used for different targets. Assays were performed at 10 μL final volume with simulated swab samples (Flu A, Flu B, and RSV virus, respectively, spiked in optimized urea lysis buffer (the sample processing buffer used in assay (B) of FIG. 9 with RNase A added). The NTC and virus copy number per reaction were as indicated. The amplification reactions were based on wet RT-LAMP.

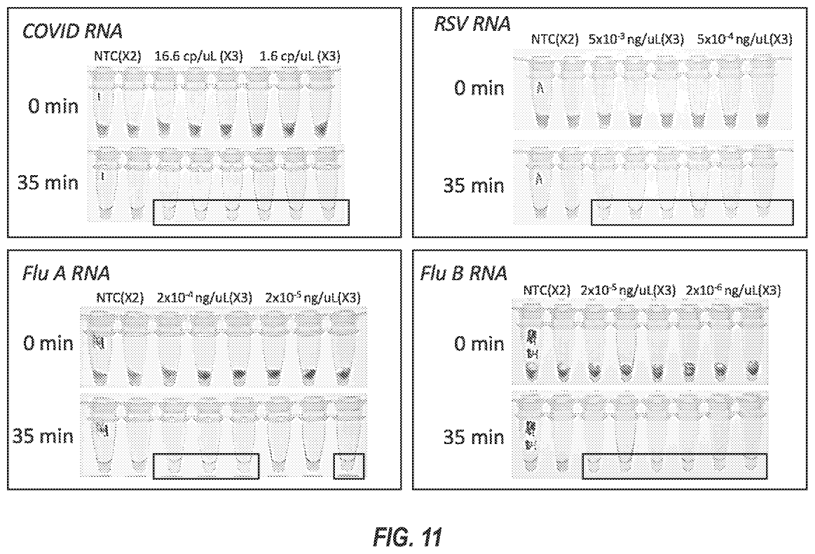

FIG. 11 illustrates results of a unified-one-pot assay used for different targets, with the master mix provided as one-pot lyophilized beads and the sample processing buffer provided as the optimized urea lysis buffer (the sample processing buffer used in assay (B) of FIG. 9). Assays were performed by rehydrating the respective one-pot lyophilized bead to 15 μL final assay volume with target RNA. The NTC and amount of target RNA added per reaction were as indicated.

FIG. 12 illustrates results of unified-one-pot assays based on different RT-LAMP master mixes and colorimetric readout indicators. Assay (A) included a commercial low-buffered master mix A with phenol red indicator. Assay (B) included a formulated, low-buffered master mix with phenol red indicator. Assay (C) included a commercial master mix B with malachite green indicator. Assay (D) included a commercial master mix C with phenol red indicator. The amplification reactions of assays A-C were performed with wet RT-LAMP. The amplification reaction of assay D was performed with one-pot lyophilized RT-LAMP subjected to thermal stability test at 35° C. for 2 hours.

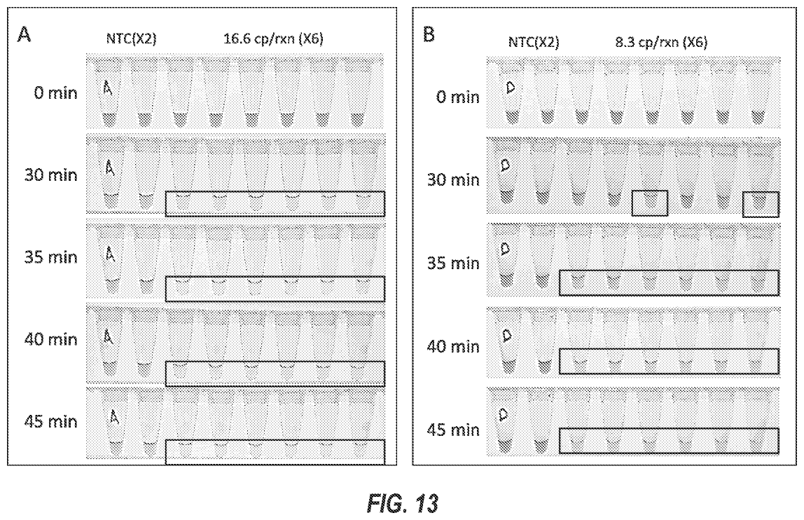

FIG. 13 illustrates results of unified-one-pot assays based on different RT-LAMP reaction mechanisms. Assay (A) utilized pH-dependent RT-LAMP based on detection of hydrogen ions (phenol red used as readout indicator in this example). Assay (B) utilized pH-independent RT-LAMP based on detection of pyrophosphate (5-Bromo-PAPS and MnCl₂ used as readout indicator in this example). Assays were performed at 15 μL final volume with simulated swab samples (SARS-CoV-2 virus spiked in lysis buffer with RNase A added). The NTC and virus copy number per reaction were as indicated. The amplification reactions were based on wet RT-LAMP.

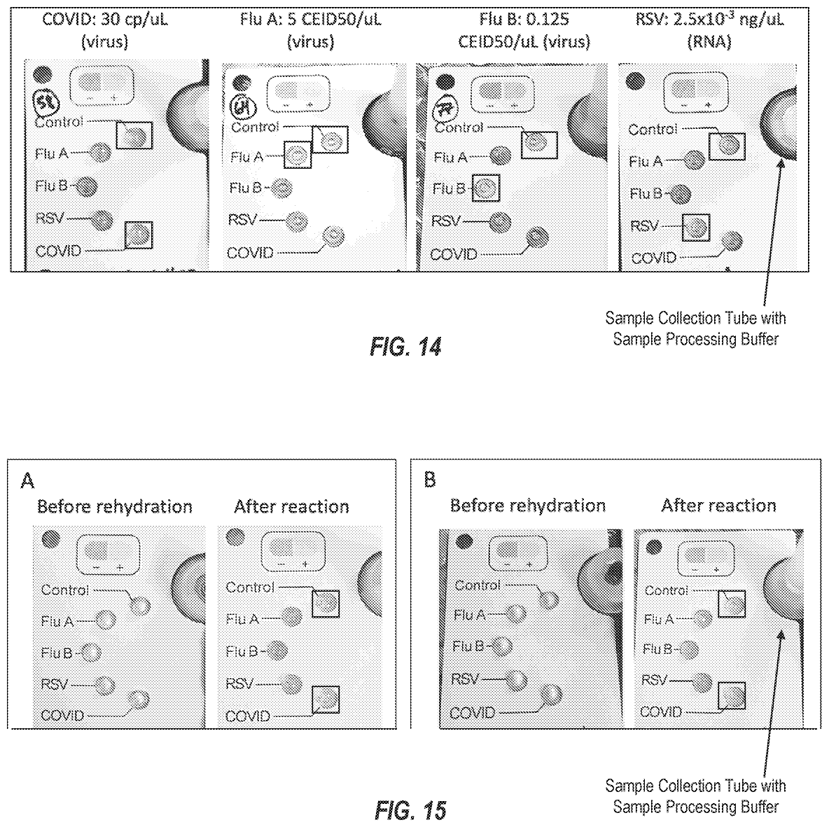

FIG. 14 illustrates results of unified-one-pot assays carried out on exemplary assay devices configured for simultaneously assaying different targets, with master mixes provided as one-pot lyophilized beads (included in the respective reaction chambers as shown) and the sample processing buffer provided as the optimized urea lysis buffer (the sample processing buffer used in assay (B) of FIG. 9). The sample processing buffers were included in sample collection tubes (as indicated), which were microfluidically connected to the one-pot lyophilized beads. Assays were performed with real nasal swabs spiked with target virus or RNA. The amount of spiked virus or RNA as indicated. As shown, each assay successfully indicated presence of the target nucleic acid, and generated a correct positive control, without generating a false positive for any off-target nucleic acids.

FIG. 15 illustrates results of stability testing of the unified-one-pot assay using the exemplary assay devices configured for simultaneously assaying different targets. Devices with one-pot lyophilized beads and sample collection tubes prefilled with sample processing buffer were both subjected to real-time or accelerated stability test at the following conditions: (A) Room temperature for 10 weeks; (B) 45° C. for 9 days. Assays were performed with real nasal swabs spiked with SARS-CoV-2 virus at 30 copies/4. As shown, both assays accurately indicated presence of SARS-CoV-2 virus, with correct positive control, and without generating a false positive for any off-target nucleic acids.

### Additional Terms & Definitions

When component amounts are described herein according to percentages, it will be understood that the percentages refer to % (w/v) unless specified otherwise.

It should be understood that for any given element or component of a described embodiment, any of the possible alternatives listed for that element or component may be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Any headings and subheadings used herein are for organizational purposes only and are not meant to be used to limit the scope of the description or the claims.

Any reference disclosed herein shall be understood to be incorporated herein by reference even if the words “incorporated” and “reference” are not expressly recited with respect to the references.

As used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

The embodiments disclosed herein should be understood as comprising/including disclosed components and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, a sample processing buffer may essentially omit or completely omit surfactants, reducing/denaturing agents, nuclease inhibitors, buffering salts, and/or chelating agents not specifically disclosed.

An embodiment that “essentially omits” a component may include trace amounts and/or non-functional amounts of the component. For example, an “essentially omitted” component may be included in an amount no more than 2%, no more than 1%, no more than 0.1%, or no more than 0.01% by total weight of the composition.

A composition that “completely omits” a component does not include a detectable amount of the component (i.e., does not include an amount above any inherent background signal associated with an appropriate testing instrument) when analyzed using standard compositional analysis techniques such as, for example, microscopy imaging techniques, chromatographic techniques (e.g., thin-layer chromatography (TLC), gas chromatography (GC), liquid chromatography (LC)), or spectroscopy techniques (e.g., Fourier transform infrared (FTIR) spectroscopy).

### REFERENCES

(1) Oliveira, B. B.; Veigas, B.; Baptista, P. V. Isothermal Amplification of Nucleic Acids: The Race for the Next “Gold Standard.” Front. Sens. 2021, 2.
(2) Li, B.; Ellington, A. D.; Chen, X. Rational, Modular Adaptation of Enzyme-Free DNA Circuits to Multiple Detection Methods. Nucleic Acids Res. 2011, 39 (16), el 10. https://doi.org/10.1093/nar/gkr504.
(3) Li, B.; Chen, X.; Ellington, A. D. Adapting Enzyme-Free DNA Circuits to the Detection of Loop-Mediated Isothermal Amplification Reactions. Anal. Chem. 2012, 84 (19), 8371-8377. https://doi.org/10.1021/ac301944v.
(4) Chen, J.; Tang, L.; Chu, X.; Jiang, J. Enzyme-Free, Signal-Amplified Nucleic Acid Circuits for Biosensing and Bioimaging Analysis. Analyst 2017, 142 (17), 3048-3061. https://doi.org/10.1039/C7AN00967D.
(5) Yin, P.; Choi, H. M. T.; Calvert, C. R.; Pierce, N. A. Programming Biomolecular Self-Assembly Pathways. Nature 2008, 451 (7176), 318-322. https://doi.org/10.1038/nature06451.
(6) Li, J.; Hu, X.; Wang, X.; Yang, J.; Zhang, L.; Deng, Q.; Zhang, X.; Wang, Z.; Hou, T.; Li, S. A Novel One-Pot Rapid Diagnostic Technology for COVID-19. Anal. Chim. Acta 2021, 1154, 338310. https://doi.org/10.1016/j.aca.2021.338310.
(7) Shedlovskiy, D.; Shcherbik, N.; Pestov, D. G. One-Step Hot Formamide Extraction of RNA from Saccharomyces Cerevisiae. RNA Biol. 2017, 14 (12), 1722-1726. https://doi.org/10.1080/15476286.2017.1345417.
(8) Chomczynski, P. Solubilization in Formamide Protects RNA from Degradation. Nucleic Acids Res. 1992, 20 (14), 3791-3792. https://doi.org/10.1093/nar/20.14.3791.
(9) Earl, C. C.; Smith, M. T.; Lease, R. A.; Bundy, B. C. Polyvinylsulfonic Acid: A Low-Cost RNase Inhibitor for Enhanced RNA Preservation and Cell-Free Protein Translation. Bioengineered 2018, 9 (1), 90-97. https://doi.org/10.1080/21655979.2017.1313648.
(10) Hayden, A.; Kuentzel, M.; Chittur, S. V. Rapid, Affordable, and Scalable SARS-CoV-2 Detection From Saliva. J Biomol. Tech. JBT 2021, 32 (3), 148-157. https://doi.org/10.7171/j bt.21-3203-01 O.
(11) Ranoa, D. R. E.; Holland, R. L.; Alnaji, F. G.; Green, K. J.; Wang, L.; Brooke, C. B.; Burke, M. D.; Fan, T. M.; Hergenrother, P. J. Saliva-Based Molecular Testing for SARS-CoV-2 That Bypasses RNA Extraction. bioRxiv Jun. 18, 2020, p 2020.06.18.159434. https://doi.org/10.1101/2020.06.18.159434.
(12) Tanner, N. A.; Zhang, Y.; Evans, T. C. Visual Detection of Isothermal Nucleic Acid Amplification Using PH-Sensitive Dyes. BioTechniques 2015, 58 (2), 59-68. https://doi.org/10.2144/000114253.
(13) Zhang, Y.; Hunt, E. A.; Tamanaha, E.; Correa, I. R.; Tanner, N. A. Improved Visual Detection of DNA Amplification Using Pyridylazophenol Metal Sensing Dyes. Commun. Biol. 2022, 5 (1), 1-9. https://doi.org/10.1038/s42003-022-03973-x.
(14) Szobi, A.; Buranovska, K.; Vojtaggakova, N.; Lovigek, D.; Ozbasak, H. O.; Szeibeczederova, S.; Kapustian, L.; Hudkova, Z.; KovaCova, V.; Drobna, D.; Putaj, P.; Birova, S.; (irkova, I.; (arneckY, M.; Kilian, P.; Jurkkek, P.; (abanova, V.; Borgova, K.; Slavikova, M.; Vanova, V.; Klempa, B.; (ekan, P.; Paul, E. D. Vivid COVID-19 LAMP Is an Ultrasensitive, Quadruplexed Test Using LNA-Modified Primers and a Zinc Ion and 5-Br-PAPS Colorimetric Detection System. Commun. Biol. 2023, 6 (1), 233. https://doi.org/10.1038/s42003-023-04612-9.
(15) Fukushima, Y.; Aikawa, S. Colorimetric Detection of Homocysteine by a Pyridylazo Dye-Based Cu2+ Complex via Indicator Displacement Mechanism. Anal. Biochem. 2021, 621, 114185. https://doi.org/10.1016/j.ab.2021.114185.
(16) Inoue, K.; Aikawa, S.; Sakamaki, M.; Fukushima, Y. Colorimetric Co2+ Sensor Based on an Anionic Pyridylazo Dye and a Cationic Polyelectrolyte in Aqueous Solution: Colorimetric Co2+ Sensor. Polym. Int 2018, 67 (12), 1589-1594. https://doi.org/10.1002/pi.5682.
(17) Fukushima, Y.; Aikawa, S. Colorimetric Detection of Mnii Based on a Mixture of an Anionic Pyridylazo Dye and a Cationic Polyelectrolyte in Aqueous Solution. Color. Technol. 2020, 136 (5), 450-456. https://doi.org/10.1111/cote.12489.
(18) Lucchi, N. W.; Ljolje, D.; Silva-Flannery, L.; Udhayakumar, V. Use of Malachite Green-Loop Mediated Isothermal Amplification for Detection of Plasmodium Spp. Parasites. PLOS ONE 2016, 11 (3), e0151437. https://doi.org/10.1371/journal. pone. 0151437.
(19) Suarez, G. D.; Suarez, D. A.; Kiu Tang, Y. Y.; Zhang, J.-X.; Li, J.; Nagl, S.; Cheung, P. P.-H. Uncovering Mechanisms of RT-LAMP Colorimetric SARS-CoV-2 Detection to Improve Assay Reliability. Anal. Methods 2022, 14 (4), 378-382. https://doi.org/10.1039/D1AY01395E.
(20) Thapa, J.; Maharjan, B.; Malla, M.; Fukushima, Y.; Poudel, A.; Pandey, B. D.; Hyashida, K.; Gordon, S. V.; Nakajima, C.; Suzuki, Y. Direct Detection of Mycobacterium Tuberculosis in Clinical Samples by a Dry Methyl Green Loop-Mediated Isothermal Amplification (LAMP) Method. Tuberculosis 2019, 117, 1-6. https://doi.org/10.1016/j.tube.2019.05.004.
(21) Fischbach, J.; Xander, N. C.; Frohme, M.; Glokler, J. F. Shining a Light on LAMP Assays' A Comparison of LAMP Visualization Methods Including the Novel Use of Berberine. BioTechniques 2015, 58 (4), 189-194. https://doi.org/10.2144/000114275.
(22) Pang, B.; Yao, S.; Xu, K.; Wang, J.; Song, X.; Mu, Y.; Zhao, C.; Li, J. A Novel Visual-Mixed-Dye for LAMP and Its Application in the Detection of Foodborne Pathogens. Anal. Biochem. 2019, 574, 1-6. https://doi.org/10.1016/j.ab.2019.03.002.
(23) Hayashida, K.; Kajino, K.; Hachaambwa, L.; Namangala, B.; Sugimoto, C. Direct Blood Dry LAMP: A Rapid, Stable, and Easy Diagnostic Tool for Human African Trypanosomiasis. PLoS Negl. Trop. Dis. 2015, 9 (3), e0003578. https://doi.org/10.1371/j ournal. pntd. 0003578.

1. A method for performing a molecular diagnostic test that combines sample processing, nucleic acid amplification, and result readout in a single unified-one-pot reaction, the method comprising:
mixing a sample with a sample processing buffer to form a sample mixture;
adding the sample mixture or portion thereof directly to a nucleic acid amplification master mix within a reaction volume to form a reaction mixture; and
subjecting the reaction mixture within the reaction volume to thermal incubation for a period of time, during which (i) the sample is processed, (ii) target nucleic acids are released and amplified, and (iii) a readout indicator included within the master mix is activated.

2. The method of claim 1, wherein the method omits a step of heating the sample mixture prior to adding the sample mixture to the nucleic acid amplification master mix.

3. The method of claim 1, wherein the sample is a lower nasal swab sample, nasopharyngeal swab sample, gingival swab sample, buccal swab sample, gargle sample, sputum sample, saliva sample, environmental sample, veterinary sample, or agricultural/food sample.

4. The method of claim 1, wherein the target nucleic acid is associated with a pathogen.

5. The method of claim 1, wherein the same sample processing buffer functions to enable one or more of sample elution, sample inactivation, sample lysis, nucleases inhibition, nucleic acid extraction, nucleic acid purification, and nucleic acid stabilization.

6. The method of claim 1, wherein the sample processing buffer comprises:
a surfactant comprising Tween 20, Tween 80, Triton X-100, Triton X-114, NP-40, Igepal CA-630, CHAPS, and/or SDS;
a reducing/denaturing agent comprising DTT, TCEP, urea, GuHC1, GITC, and/or formamide;
optionally, a nuclease inhibitor comprising proteinase K, murine RNase inhibitor, human placenta RNase inhibitor, VSA, PVSA, ACP, RNasin Ribonuclease Inhibitor (native or recombinant), RNasin Plus Ribonuclease Inhibitor, RiboLock RNase Inhibitor, SUPERase In, RNaseOUT, and/or RNAsecure;
optionally, a chelating agent comprising EDTA; and
optionally, a buffering salt comprising Tris, Tris-HCl, TE, TAE, TBE, a HCl solution, a NaOH solution and/or a KOH solution.

7. The method of claim 6, wherein the reducing/denaturing agent comprises urea and/or formamide.

8. The composition of claim 1, wherein the nucleic acid amplification is isothermal, and the unified-one-pot reaction takes place inside a single reaction vessel at a single target incubation temperature.

9. The method of claim 1, wherein the nucleic acid amplification is not isothermal, and the unified-one-pot reaction takes place inside a single reaction vessel subjected to suitable thermocycling conditions.

10. The method of claim 1, wherein the sample mixture or portion thereof is added to the nucleic acid amplification master mix by pipetting, microfluidics, microcapillaries, wicking by a porous media or material, or combination thereof.

11. The method of claim 1, wherein the nucleic acid amplification master mix has an air-dried or lyophilized form, and wherein adding the sample mixture or portion thereof to the nucleic acid amplification master mix reconstitutes the reaction mixture to a desired total reaction volume.

12. The method of claim 1, wherein the nucleic acid amplification method is selected from LAMP, RT-LAMP, DAMP, CPA, SDA, RCA, RPA, HDA, NASBA, MDA, WGA, GEAR, EXPAR, NEAR, SPIA, ICAN, PHAMP, SMART, BAD AMP, CRISDA, HCR, CHA, EHA, EDC, TMSD, PCR, RT-PCR, qPCR, RT-qPCR, or a combination thereof.

13. The method of claim 1, wherein the readout indicator functions according to: pH, turbidity; fluorescence; nanomaterials; detection of pyrophosphate; a lateral flow strip/dipstick mechanism; gel/capillary electrophoresis; microfluidics; microarrays; electrochemical sensors; molecular transducers; or a combination thereof.

14. The method of claim 1, wherein the readout indicator comprises:
a pH indicator, optionally selected from Phenol Red, Neutral Red, Cresol Red, Cresol Purple, Thymol Blue, Bromothymol Blue, Bromophenol Blue, Litmus, Chlorophenol Red, Dichlorofluorescein, Methyl Red, Bromocresol Purple, Naphtholphthalein, and/or Cresolphthalein;
a metal indicator that senses metal ions such as Mg²⁺, Mn²⁺, Zn²⁺, Cu²⁺, Co²⁺, Cd²⁺, Fe²⁺, Ni²⁺, Hg²⁺, Pb²⁺, such as a composition comprising one or more of
Hydroxynaphthol Blue,
Eriochrome Black T,
Calcein,
pyridylazophenol dye such as 2-(5-Bromo-2-pyridylazo)-54N-propyl-N-(3-sulfopropyl) aminolphenol (5-Bromo-PAPS) or 2-(5-Nitro-2-pyridylazo)-5-[N-n-propyl-N-(3-sulfopropyl)amino] phenol (5-Nitro-PAPS);
a fluorescent/colorimetric DNA binding dye such as SYBR Gold, SYBR Safe, Leuco Crystal Violet, Malachite Green, Methyl Green, EvaGreen, SYTO 9; and/or
a nanoparticle-based indicator such as gold nanoparticles.

15. The method of claim 1, wherein the master mix comprises:
MgSO₄ or MgCl₂, optionally included at 4 mM to 8 mM;
(NH₄)₂SO₄ or (NH₄)₂Cl₂, optionally included at 10 mM;
dNTP mix, optionally included at 1 mM to 2 mM;
DNA polymerase, the DNA polymerase optionally comprising Bst 2.0 or Bst 2.0 WarmStart DNA Polymerase;
Tween 20 or Triton-100, optionally at pH 8.8 and optionally included at 0.1%;
Tris-HCl, optionally at pH 8.8 and optionally included at 2 mM to 20 mM, or Tris or TE;
KCl, optionally included at 10 mM to 50 mM;
the master mix optionally further comprising
betaine, optionally included at 0.8 mM;
primers for specific amplification of a target nucleic acid sequence from said sample, the primers optionally comprising 0.8 μM F3 primer, 0.8 μM B3 primer, 1.6 μM FIP primer, 1.6 μM BIP primer, and optionally 0.4 μM LF primer and/or 0.4 μM LB primer;
reverse transcriptase;
GuHCl;
Antarctic Thermolabile Uracil-DNA-glycosylase (UDG) and dUTP;
one or more reaction enhancers comprising crowding agents, dsDNA destabilizers, dsDNA stabilizers, enzyme stabilizers, template blockers, and/or oligonucleotide modifications or analogs; and
one or more excipients comprising sucrose, trehalose, dextran, pullulan, lactose, glucose, raffinose, mannitol, sorbitol, glycine, histidine, arginine, gelatin, dextrose, hydroxyethyl starch, poly(ethylene glycol), poly(propylene glycol), and/or poly(vinyl alcohol).

16. The method of claim 1, wherein the sample processing buffer is weakly pH-buffered, fully pH-buffered, or adjusted to a pH suitable for the nucleic amplification reaction and its readout chemistry.

17. The method of claim 16, wherein the readout indicator comprises phenol red, optionally included at a concentration of 50 μM to 100 μM, and the sample processing buffer is weakly buffered and adjusted to a pH of about 8.0-8.2.

18. The method of claim 16, wherein the readout indicator comprises 5-Bromo-PAPS or 5-Nitro-PAPS, optionally at a concentration of about 50 μM to 200 μM, and Mn²⁺ or Zn²⁺, optionally at a concentration of about 50 μM to 300 μM, and the sample processing buffer is fully buffered.

19. A composition for performing a molecular diagnostic test in a unified-one-pot format combining sample processing and nucleic acid amplification, the composition comprising:
(i) a sample processing buffer comprising
a surfactant comprising Tween 20, Tween 80, Triton X-100, Triton X-114, NP-40, Igepal CA-630, CHAPS, and/or SDS;
a reducing/denaturing agent comprising DTT, TCEP, urea, GuHC1, GITC, and/or formamide;
optionally, a nuclease inhibitor comprising proteinase K, murine RNase inhibitor, human placenta RNase inhibitor, VSA, PVSA, ACP, RNasin Ribonuclease Inhibitor (native or recombinant), RNasin Plus Ribonuclease Inhibitor, RiboLock RNase Inhibitor, SUPERase In, RNaseOUT, and/or RNAsecure;
optionally, a chelating agent comprising EDTA; and
optionally, a buffering salt comprising Tris, Tris-HCl, TE, TAE, TBE, a HCl solution, a NaOH solution and/or a KOH solution;
(ii) a nucleic acid amplification master mix; and
(iii) a readout indicator.

20. A kit for performing a molecular diagnostic test in a unified-one-pot format, the kit comprising:
a sample collection device;
a first vessel comprising a sample processing buffer;
a second vessel comprising a frozen, air-dried, or lyophilized nucleic acid amplification master mix for nucleic acid amplification and result readout;
a device for transferring a portion of a sample mixture from the first vessel into the second vessel;
optionally, a heat source that generates heat with a temperature profile suitable for performing a unified-one-pot sample processing and nucleic acid amplification reaction; and
optionally, a reader device to aid in interpretation and report of the test result.
